(19) United States
(12) Patent Application Publication
(10) Pub. No.: US 2004/0050721 A1
(43) Pub. Date: Mar. 18, 2004

(54) Suture retainer package

(76) Inventors: Mark Roby, Killingworth, CT (US); John Kennedy, Guilford, CT (US)

Correspondence Address: Mark Farber, C/O U.S. Surgical, a division of, TYCO HEALTHCARE GROUP LP, 150 Glover Avenue, Norwalk, CT, 06856, US

(21) Appl. No.: 10/618,806

(22) Filed: Jul. 14, 2003

Related U.S. Application Data
(60) Provisional application No. 60/396,943, filed on Jul. 17, 2002.

(51) Int. Cl. A61B 17/06

(52) U.S. Cl. 206/63.3

(57) ABSTRACT

The present disclosure relates to packages for retaining a needle-suture combination. According to one aspect of the present disclosure, the package includes a moldable base portion; a moldable cover portion fabricated from moldable material, the cover portion being integrally connected to the base portion via a hinge, wherein at least one of the base portion and the cover portion is configured and adapted to retain a suture of a needle-suture combination; and a needle park disposed on at least one of the base portion and the cover portion, the needle park being configured and adapted to retain a needle of the needle-suture combination therein.

### CROSS-REFERENCE TO RELATED APPLICATION

The present application claims the benefit of and priority to U.S. Provisional Patent Application Serial No. 60/396,943, filed on Jul. 17, 2002, the entire contents of which are hereby incorporated by reference.

### TECHNICAL FIELD

The present disclosure relates to packages for surgical sutures, and more particularly to packages for retaining, storing, and dispensing a surgical needle-suture combination.

### BACKGROUND OF RELATED ART

Various types and styles of suture packages have been developed to hold surgical needles and associated lengths of suture for use during a surgical operation. Generally, a suture retainer should be constructed to adequately secure a needle and suture while providing easy withdrawal during use. It is also preferable to minimize permanent deformation of the suture by kinking or bending during storage and removal from the suture retainer.

Suture retainers typically include a folded pouch containing a single needle and suture combination. To access the needle and suture, the package is at least partially unfolded and the needle/suture is removed by hand. The suture is typically wound in an oval or figure “8” pattern within the retainer. The needle can be secured in a slot or die cut formed in the pouch or, in the alternative, can piercingly engage a foam strip affixed to the pouch.

Another type of retainer is of molded construction, such as, for example, the retainer disclosed in U.S. Pat. No. 5,154,283 to Brown. The retainer described in the Brown patent includes a molded cover member having a spiraled passageway formed therein to accommodate a suture and a cover sheet adhered to the molded member to close the passageway.

Other types of suture retainers are known and disclosed in the art. For example, commonly assigned U.S. Pat. No. 5,121,836, herein incorporated by reference, discloses a multi-panel suture retainer for an armed surgical suture. The suture retainer has a needle retaining panel, a front cover panel, a suture winding panel, and a fold over panel. Apertures for loading pins facilitate the loading of the suture in a “figure 8”, or lemniscate type pattern. The lemniscate pattern includes a central crossover point where the suture strand crosses over itself. While this package and suture pattern has been used successfully for years, it is desirable to eliminate suture crossover points to reduce potential wear during shipment and storage.

Accordingly, the need exists for packages for retaining a plurality of sutures therein which minimizes damage to the suture and which minimizes the amounts of overlapping and/or crossover (i.e., “zig-zagging”) of the suture in the package.

### SUMMARY

The present disclosure relates to packages for retaining a needle-suture combination. According to one aspect of the present disclosure, the package includes a moldable base portion; a moldable cover portion, the cover portion being integrally connected to the base portion via a hinge, wherein at least one of the base portion and the cover portion is configured and adapted to retain a suture of a needle-suture combination; and a needle park disposed on at least one of the base portion and the cover portion, the needle park being configured and adapted to retain a needle of the needle-suture combination therein.

The hinge preferably integrally interconnects the base portion with the cover portion. It is envisioned that the hinge is a living hinge.

The base portion and the cover portion are correspondingly shaped. Preferably, the base portion and the cover portion are ovular in shape.

The base portion and/or the cover portion includes at least one rail integrally formed therewith and defines a suture race around the circumference of the package. The base portion and/or the cover portion includes at least one aperture formed therein for passage of the suture of the needle-suture combination therethrough.

Preferably, the base portion and the cover portion are fabricated from plastic.

According to another aspect of the present disclosure the package includes a base portion having a bottom wall; an outer perimeteral wall extending therearound; a rail provided on the bottom wall and extending substantially therearound, the rail and the outer perimetral wall defining a race therebetween; and an aperture formed in the bottom wall for passage of a suture retained in the suture race from the package. The package further including a cover hingedly connected to the base portion. Preferably, the hinge is a living hinge.

In certain embodiments, the rail is provided with at least one opening formed along the length thereof. It is envisioned that the package can be provided with a suture wound around the rail and within the suture race. Preferably, the suture includes a distal end extending through the at least one opening in the rail and through the aperture in the bottom wall.

The outer perimeteral wall can include at least one tab extending therefrom and configured and dimensioned to selectively engage an edge of the cover.

Preferably, the package is formed of plastic. In certain embodiments it is contemplated that a lubricious material can be provided in the suture race.

It is envisioned that the package can further include at least one needle park formed in the bottom wall of the base portion, the needle park being configured and adapted to retain a needle therein.

The present disclosure further relates to a method of loading a package for retaining a needle-suture combination. The method includes the steps of providing a package for retaining a needle-suture combination. The package includes a molded base portion having a hinge-side edge and a free edge; a molded cover portion having a hinge-side edge and a free edge, the cover portion being integrally connected to the base portion along the hinge-side edges; and a needle park disposed on at least one of the base portion and the cover portion, the needle park being configured and adapted to retain a needle of the needle-suture combination therein. The method further includes the steps of loading the package with a suture of the needle-suture combination; and approximating the free edges of the base portion and the cover portion toward one another.

It is envisioned that the free ends of the base portion and the cover portion are snappingly engagable with one another.

These and other advantages of the disclosure will become apparent to those skilled in the art from the foregoing general description of the following detailed disclosure, and from practice of the invention.

### BRIEF DESCRIPTION OF THE DRAWINGS

Various embodiments are described herein with reference to the drawings, wherein:

FIG. 1 is a top perspective view of a suture retainer package in accordance with the present disclosure.

### DETAILED DESCRIPTION OF PREFERRED EMBODIMENTS

As used herein, the terms “distal” and “proximal”, “above” and “below”, “up” and “down”, “left” and “right” and similar such terms are used relative to each other and not to an external fixed frame of reference.

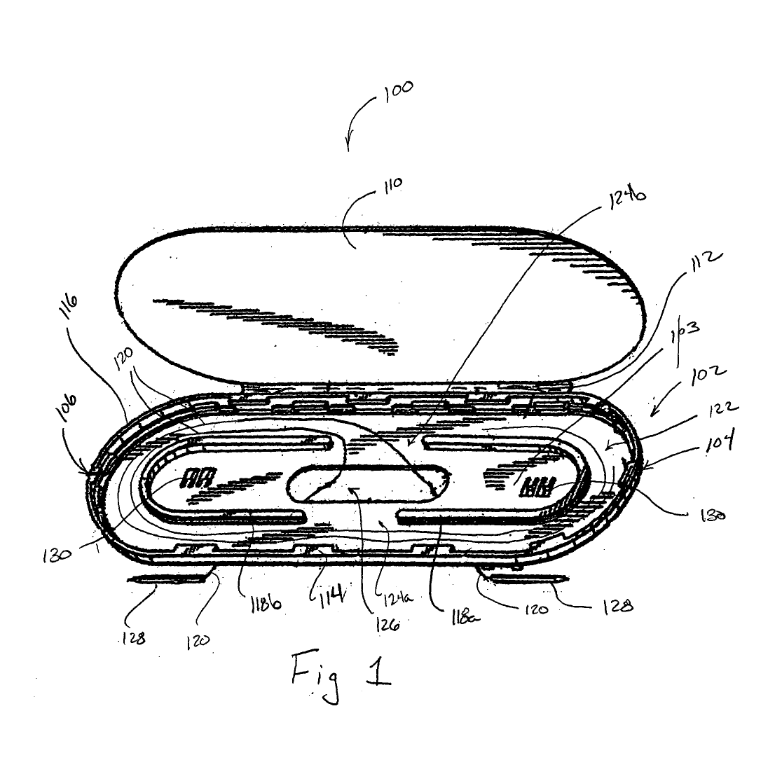

With reference to FIG. 1, there is illustrated a preferred embodiment of a suture retainer package, generally designated with reference numeral 100. Package 100 is particularly configured to accommodate a length of suture for removal therefrom without undesirable deformation of the suture, and to retain a needle attached to the suture in a readily accessible position adjacent the suture package wherein the needle is grasped by hand or needle graspers as is known in the art.

Suture retainer package 100 is generally flat and ovular in shape and includes a bottom or base portion 102 including a bottom wall 103 having a first arcuate end 104 and a second arcuate end 106 each having substantially the same radius. Base portion 102 is preferably formed from a rigid plastic material which can be molded into shape, although other materials known in the art can be used.

Base portion 102 includes an outer perimeteral wall 116 extending therearound. Base portion 102 further includes a pair of inner rails 118a, 118b formed in a top surface of bottom wall 103 and extending substantially therearound. Preferably, rails 118a, 118b are spaced a distance from perimeteral wall 116 and thereby define a suture race 122 around base portion 102. Rails 118a, 118b are spaced from one another and define a pair of rail openings 124a, 124b therebetween.

Suture retainer package 100 further includes a top or cover portion 110 having substantially the same shape as base portion 102. Cover portion 110 is preferably connected to base portion 102 along a side thereof via a web portion defining a hinge 112, in the manner of a clam shell top. Preferably, hinge 112 is a living hinge. Cover portion 110 can be fabricated from a moldable rigid plastic or the like. By making base portion 102 and cover portion 110 integral with one another, the possibility of cover portion 110 and base portion 102 becoming separated from one another and lost relative to one another, is reduced and/or eliminated.

Preferably, cover portion 110 is selectively secured in place against base portion 102 via closure elements (e.g., flaps or tabs) 114 extending inwardly from outer wall 116 of base portion 102, preferably, on a side opposite living hinge 112. In use, when cover portion 110 is brought against base portion 102, the free side of cover portion 110 snaps into base portion 102, i.e., under tabs 114.

Base portion 102 further includes at least one aperture 126 formed in bottom wall 103 thereof for passage of suture 120 therethrough, as will be described in greater detail below. It is contemplated that either base portion 102, cover potion 110 or both base portion 102 and cover portion 110 are each provided with at least one aperture or opening formed therein to permit loading or removal of sutures 120 therefrom.

It is further contemplated that a lubricating or slip agent can be provided on the suture contacting surface of base portion 102 which facilitates movement or passage of sutures 120 within retainer package 100 and in particular around suture race 122.

Preferably, suture 120 is retained in package 100 such that suture 120 is wound around rails 118a, 118b and the distal-most end of suture 120 pass between rail openings 124a, 124b and out through aperture 126.

As seen in FIG. 1, the preferred suture package 100 is provided with a needle 128 affixed to an end of suture 120. At least one needle park 130 can be provided on base portion 102 for holding needle(s) 128 in place during storage and/or shipping.

While a single suture race 122 defined by rails 118a, 118b has been shown and described, it is envisioned that multiple races can be provided by multiple layers of rails.

Exemplary suture retaining packages are disclosed and described in commonly assigned U.S. Pat. No. 5,733,293 to Scirica, et al. and U.S. Pat. No. 6,260,699 to Kaplan et al., the entire contents of each of which are incorporated herein by reference.

It will be understood that various modifications may be made to the embodiments described herein. For example, it is envisioned that package 100 is entirely fabricated from a rigid moldable plastic. In other words, base portion 102, including all of the features thereof, cover portion 110 and living hinge 112 can all be molded as a single unitary piece of rigid moldable plastic. Preferably, package 100 is formed from a single piece of clear rigid moldable plastic. In this manner, the user can visually ascertain whether additional suture 120 is present in package 100 without having to open package 100.

While rails 118a, 118b have been shown as being formed in base portion 102, it is envisioned that rails 118a, 118b or other rails can be formed in cover portion 110. In addition, while cover portion 110 is shown and described as engaging tabs 114 extending from outer perimeteral wall 116, it is envisioned that cover portion 110 can be provided with closure elements formed along the outer edge thereof for engaging base portion 102.

Therefore the above description should not be construed as limiting, but merely as exemplifications of preferred embodiments. Those skilled in the art will envision other modifications within the scope and spirit of the claims appended hereto.

What is claimed is:

1. A package for retaining a needle-suture combination, the package comprising:
a molded base portion;
a molded cover portion, the cover portion being integrally connected to the base portion via a hinge, wherein at least one of the base portion and the cover portion is configured and adapted to retain a suture of a needle-suture combination; and
a needle park disposed on at least one of the base portion and the cover portion, the needle park being configured and adapted to retain a needle of the needle-suture combination therein.

2. The package according to claim 1, wherein the hinge integrally interconnects the base portion with the cover portion.

3. The package according to claim 2, wherein the hinge is a living hinge.

4. The package according to claim 1, wherein the base portion and the cover portion are correspondingly shaped.

5. The package according to claim 4, wherein the base portion and the cover portion are ovular in shape.

6. The package according to claim 2, wherein at least one of the base portion and the cover portion includes at least one rail integrally formed therewith and defining a suture race around the circumference of the package.

7. The package according to claim 2, wherein at least one of the base portion and the cover portion includes at least one aperture formed therein for passage of the suture of the needle-suture combination therethrough.

8. The package according to claim 1, wherein the base portion and the cover portion are formed from plastic.

9. A package for retaining a needle-suture combination, the package comprising:
a base portion having:
a bottom wall;
an outer perimeteral wall extending therearound;
a rail provided on the bottom wall and extending substantially therearound, the rail and the outer perimetral wall defining a race therebetween; and
an aperture formed in the bottom wall for passage of a suture retained in the suture race from the package; and
a cover connected to the base portion via a hinge.

10. The package of claim 9, wherein the hinge is a living hinge.

11. The package of claim 9, the rail is provided with at least one opening formed along the length thereof.

12. The package of claim 9, wherein the outer perimeteral wall includes at least one tab extending therefrom and configured and dimensioned to selectively engage an edge of the cover.

13. The package of claim 11, further including a suture wound around the rail and within the suture race, the suture including a distal end extending through the at least one opening in the rail and through the aperture in the bottom wall.

14. The package of claim 9, wherein the package is formed of plastic.

15. The package of claim 9, further including a lubricious material provided in the suture race.

16. The package of claim 9, further includes at least one needle park formed in the bottom wall of the base portion, the needle park being configured and adapted to retain a needle therein.

17. A method of loading a package for retaining a needle-suture combination, the method including the steps of:
providing a package for retaining a needle-suture combination, the package including:
a molded base portion having a hinge-side edge and a free edge;
a molded cover portion having a hinge-side edge and a free edge, the cover portion being integrally connected to the base portion along the hinge-side edges; and
a needle park disposed on at least one of the base portion and the cover portion, the needle park being configured and adapted to retain a needle of the needle-suture combination therein;
loading the package with a suture of the needle-suture combination; and
approximating the free edges of the base portion and the cover portion toward one another.

18. The method according to claim 17, wherein the free ends of the base portion and the cover portion are snappingly engagable with one another.
